(19) United States
(12) Patent Application Publication
(10) Pub. No.: US 2007/0031880 A1
(43) Pub. Date: Feb. 8, 2007

(54) Chemical treatment of biological samples for nucleic acid extraction and kits therefor

(75) Inventors: Jianrong Lou, Mount Airy, MD (US); Matthew P. Collis, Seven Valleys, PA (US); Thomas L. Fort, Finksburg, MD (US)

Correspondence Address: DAVID W HIGHET VP AND CHIEF IP COUNSEL;BECTON DICKINSON AND COMPANY, 1 BECTON DRIVE, MC110, FRANKLIN LAKES, NJ, 07417-1880, US

(73) Assignee: Becton, Dickinson and Company

(21) Appl. No.: 11/544,040

(22) Filed: Oct. 6, 2006

Related U.S. Application Data
(63) Continuation of application No. 10/359,180, filed on Feb. 6, 2003, now abandoned.

(51) Int. Cl. C12Q 1/68; C07H 21/04

(52) U.S. Cl. 435/6; 536/25.4

(57) ABSTRACT

A composition and method for the purification of nucleic acid are disclosed. The composition includes at least one alkaline agent and at least one detergent. The composition preferably also includes a suspension of paramagnetic particles and an acidic solution. The method involves the use of the composition with paramagnetic particles to extract nucleic acid from a biological sample.

### FIELD OF THE INVENTION

The present invention relates to extraction, isolation or purification of nucleic acids (i.e., DNA or RNA) from plasma, whole blood and other biological samples by paramagnetic surface binding or other nucleic acid extraction methods. Extracted nucleic acid can be used for various DNA/RNA applications such as nucleic acid amplification and/or detection for the diagnosis of disease.

### BACKGROUND OF THE INVENTION

Access to cellular components such as nucleic acids is imperative to a variety of molecular biology methodologies including nucleic acid sequencing, direct detection of particular nucleic acid sequences by nucleic acid hybridization and nucleic acid sequence amplification techniques. The extraction, isolation or purification of DNA or RNA is an important step in many biochemical and diagnostic procedures. For example, the extraction and separation of nucleic acids from the complex mixtures in which they are often found is frequently necessary before other studies and procedures, e.g., cloning, sequencing, amplification, hybridization, cDNA synthesis, detection, etc., can be undertaken. The presence of large amounts of cellular or other contaminating material, e.g., proteins or carbohydrates, in such complex mixtures often impedes many of the reactions and techniques used in molecular biology. For example, plasma and whole blood are clinical samples commonly used for nucleic acid-based diagnostics. High protein levels and the high RNase/DNase levels are major obstacles for processing such samples, as well as other samples having high amounts of protein and/or RNase or DNase.

In addition, DNA may contaminate RNA preparations and vice versa. Thus, methods for the extraction, isolation or purification of nucleic acid from complex mixtures such as cells, tissues, etc. is desirable, not only from the preparative point of view, but also as part of the many methods in use today which rely on the identification of DNA or RNA, e.g., clinical diagnosis, forensic science, tissue and blood typing, detection of genetic variations, etc.

A number of methods are known for the extraction, isolation or purification of nucleic acids. Generally, such methods rely on a complex series of extraction, isolation or purification steps, which are time consuming and laborious to perform. Moreover, such methods involve the use of materials such as alcohols and other organic solvents, chaotropes and proteinases, which is disadvantageous because such materials tend to interfere with many enzymatic reactions and other downstream processing applications.

Classical methods for the extraction, isolation or purification of nucleic acid from complex starting materials such as blood, blood products, tissues or other biological samples involve lysis of the biological material by a detergent or chaotrope, possibly in the presence of protein degrading enzymes, followed by several extractions with organic solvents, e.g., phenol and/or chloroform, ethanol precipitation, centrifugation and dialysis of the nucleic acids. The purification of RNA from DNA may involve additional steps, for example, selective precipitation with LiCl or selective isolation with acidic guanidinium thiocyanate combined with phenol extraction and ethanol precipitation. Not only are such methods cumbersome and time consuming to perform, but also the relatively large number of steps required increases the risk of degradation, sample loss or cross-contamination of samples where several samples are simultaneously processed. In the case of RNA isolation, the risk of DNA contamination is relatively high. The purification of double-stranded plasmid DNA, single-stranded phage DNA, chromosomal DNA, agarose gel DNA fragments and RNA is of critical importance in molecular biology. Ideally, a method for purifying nucleic acids should be simple, rapid and require little, if any, additional sample manipulation. Nucleic acids obtained by such a method should be immediately amenable to transformation, restriction analysis, ligation or sequencing. A method capable of providing DNA or RNA of high purity is, therefore, highly desirable.

Another purification method for preparation of plasmid DNA from crude alcohol precipitates is laborious, most often utilizing CsCl gradients, gel filtration, ion exchange chromatography, and repeated alcohol precipitation steps. These methods also require considerable downstream sample preparation to remove CsCl and other salts, ethidium bromide and alcohol. A further problem with these methods is that small, negatively charged cellular components can co-purify with the DNA. Thus, the DNA can have an undesirable level of contamination.

Nucleic acids can also be purified using solid phases. Conventional solid phase extraction techniques have utilized silica-type surfaces that either (1) fail to attract and hold sufficient quantities of nucleic acid molecules to permit easy recovery, or (2) excessively adhere to the nucleic acid molecules, thereby hindering their recovery. Conventional surfaces that cause these problems include surfaces such as glass and Celite. Adequate binding of nucleic acids to these types of surfaces can be achieved only by utilizing high concentrations of chaotropes or alcohols, which are generally toxic, caustic, and/or expensive. For example, it is known that DNA will bind to crushed glass powders and to glass fiber filters in the presence of chaotropes. The chaotropic ions typically are washed away with alcohol, and the DNA is eluted with low-salt solutions or water. A serious drawback in the use of crushed glass powder is that its binding capacity is low. In addition, glass powder often suffers from inconsistent recovery, incompatibility with borate buffers and a tendency to nick large DNAs. Similarly, glass fiber filters provide a nonporous surface with low DNA binding capacity. Other silica-type surfaces, such as silica gel, hydrated and hydroxylated silica surfaces as disclosed in EP 0512767 and U.S. Pat. Nos. 5,674,997, 5,693,785 and 6,355,792, do not require chaotropic agents for surface binding.

There are numerous protocols for purifying DNA. For example, U.S. Pat. No. 4,923,978 discloses a process for purifying DNA in which a solution of protein and DNA is passed over a hydroxylated support, the protein is bound and the DNA is eluted. U.S. Pat. No. 4,935,342 discloses purification of DNA by selective binding of DNA to anion exchangers and subsequent elution. U.S. Pat. No. 4,946,952 discloses DNA isolation by precipitation with water-soluble ketones. A DNA purification procedure using chaotropes and dialyzed DNA is disclosed in U.S. Pat. No. 4,900,677.

Diatoms have also been utilized for purification of nucleic acids as evidenced by U.S. Pat. No. 5,234,809 and U.S. Pat. No. 5,075,430. U.S. Pat. No. 5,234,809 discloses a method where nucleic acids are bound to a solid phase in the form of silica particles in the presence of a chaotropic agent such as guanidinium salt and thereby separated from the remainder of the sample.

Although such methods speed up the nucleic acid separation process, there are disadvantages associated with the use of alcohols, chaotropes and other similar agents. Chaotropes are generally used at a high molarity, resulting in viscous solutions that may be difficult to work with, especially when working with RNA. Amplification procedures such as the polymerase chain reaction (“PCR”) and other enzyme based reactions are very sensitive to the inhibitory or otherwise interfering effects of alcohols and other agents. Moreover, the drying of the nucleic acid pellet, which is necessary following alcohol precipitation, and the problems associated with dissolving nucleic acids are also known to lead to artifacts in enzyme-based procedures, such as PCR. Yet a further technique utilized for purification of nucleic acids is binding to specifically adapted paramagnetic particles. Examples of such techniques may be found in references such as EP 0 446 260 B1 and U.S. Pat. No. 5,512,439, which describe monodisperse, superparamagnetic particles having a particle diameter standard deviation of less than 5%. Each particle carries a plurality of molecules of an oligonucleotide, with each oligonucleotide having a section serving as a probe for a target nucleic acid molecule of interest.

U.S. Pat. No. 4,672,040 and U.S. Pat. No. 4,695,393 describe magnetically responsive particles for use in systems to separate certain molecules. The particles have a metal oxide core surrounded by a stable silicone coating to which organic and/or biological molecules may be coupled.

U.S. Pat. No. 3,970,518 describes a method of sorting and separating a select cell population from a mixed cell population. The method utilizes small magnetic particles coated with an antibody to select cell populations.

U.S. Pat. No. 4,141,687 describes an automatic apparatus and method to assay fluid samples. The apparatus utilizes a particulate material with a reagent bound thereto. The particulate material is magnetic, and the reagent is a substance that takes part in a reaction in the reaction mixture.

U.S. Pat. No. 4,230,685 describes a method for magnetic separation of cells. The method utilizes magnetically responsive microspheres coated with staphylococcal Protein A to which antibody is bound.

U.S. Pat. No. 4,774,265 describes a process for preparing magnetic polymer particles. The particles are compact or porous polymer particles treated with a solution of iron salts.

U.S. Pat. No. 5,232,782 describes magnetizable “core-shell” microspheres having a core of a magnetizable filler and a shell of crosslinked organopolysiloxane.

U.S. Pat. No. 5,395,688 describes magnetically responsive fluorescent polymer particles having a polymeric core coated evenly with a layer of polymer containing magnetically responsive metal oxide.

International Publication No. WO 96/18731 describes a method for isolating nucleic acid from a sample using a particulate solid support and an anionic detergent.

U.S. Pat. No. 5,705,628 describes a method for DNA purification and isolation using magnetic particles with functional group-coated surfaces.

International Publication No. WO 01/46404 discloses a method for separating nucleic acid from a test sample that includes contacting the sample with a metal oxide support material and a binding buffer to form a nucleic acid/metal oxide support material complex, separating the complex from the test sample, and eluting the nucleic acid from the metal oxide support material. WO 01/46404 discloses that the buffer generally comprises a chaotropic agent and a detergent.

U.S. Pat. No. 5,973,138, the entire contents of which are incorporated herein by reference, discloses a composition that reversibly binds a nucleic acid molecule. The composition includes a paramagnetic particle in an acidic environment.

Iron oxide extraction of nucleic acid is non-specific, i.e., iron oxide binds nucleic acid irrespective of its form (RNA or DNA) or sequence. Extraction of nucleic acid with iron oxide is less efficient in highly proteinaceous mileus such as plasma. This may be attributable to

competition between nucleic acid and protein for iron oxide binding sites, (2) reduced kinetics due to higher viscosity of high protein solutions, (3) the effect of endogenous sample nucleases on nucleic acids, or (4) any combination of (1)-(3).

There is a need for improved methods of nucleic acid extraction, isolation or purification and particularly for methods that are quick and simple to perform and which avoid the use of chaotropic agents or alcohol precipitation. There is also a need for a method that permits isolation of both types of nucleic acid from the same sample.

### SUMMARY OF THE INVENTION

In order to provide a more effective and efficient technique for the extraction, isolation or purification of nucleic acids, the present invention provides a composition useful for extraction and reversible binding of a nucleic acid molecule. The composition comprises, in combination, at least one alkaline agent and at least one detergent. In a preferred embodiment, the composition also comprises a suspension of paramagnetic particles. In a more preferred embodiment, the composition further comprises an acidic solution.

The present invention also includes the composition packaged as a kit, as well as methods utilizing the composition to reversibly bind a nucleic acid molecule. The kit comprises a package unit having one or more containers of the subject composition. In some embodiments, the kit includes containers of various reagents used with the subject composition to purify and detect nucleic acid. The kit may also contain one or more of the following items: collection devices such as swabs, pH indicators and controls for processing and assaying the biological sample. Kits may include containers of reagents mixed together in suitable proportions for performing the methods in accordance with the invention. Reagent containers preferably contain reagents in unit quantities that obviate measuring steps when performing the subject methods.

The method of the present invention involves extracting and purifying nucleic acid from a biological sample comprising contacting the sample with at least one alkaline agent and at least one detergent; providing a suspension of at least one paramagnetic particle; providing an acidic solution; and combining the suspension and the acidic solution with the treated biological sample such that at least one nucleic acid molecule in the biological sample is reversibly bound to the at least one paramagnetic particle. The desired DNA or RNA may then be eluted from the at least one paramagnetic particle using the appropriate buffer, e.g., Tris, Bicine, CAPS, HEPES, water, potassium phosphate, Tricine, and assay buffers which may or may not contain DMSO and/or glycerol. The method of the present invention has the advantage over previous methods of processing of not requiring the use of caustic agents such as chaotropes and alcohols.

Other features and advantages of the present invention will be apparent from the following detailed description and also from the appended claims.

### DETAILED DESCRIPTION OF THE INVENTION

As used herein, the term “paramagnetic particles” means particles capable of having a magnetic moment imparted to them when placed in a magnetic field. Paramagnetic particles, when in such a magnetic field, are movable under the action of such a field. Such movement is useful for moving bound nucleic acid molecules for different aspects of sample processing. Thus, nucleic acid molecules bound to the paramagnetic particles can be processed as desired with different reagents and/or conditions with minimal direct contact due to the application of magnetic force.

As used herein, the terms “purifying” and “purification” include extracting/extraction and isolating/isolation.

The present inventors have discovered that treating biological samples with a combination of at least one alkaline agent and at least one detergent prior to combination with the paramagnetic particles allows protein denaturation to occur before the sample makes contact with the paramagnetic particles.

The biological sample useful in the present invention may be any material containing nucleic acid including, for example, clinical, forensic and environmental samples. The sample will generally be a biological sample that may contain any viral or cellular material, including prokaryotic and eukaryotic cells, viruses, bacteriophages, mycoplasms, protoplasts and organelles. Such biological materials may thus comprise all types of mammalian and non-mammalian animal cells, plant cells, algae including blue-green algae, fungi, bacteria and protozoa. Representative examples include whole blood and blood-derived products such as plasma and serum, urine, semen, feces, finger nails, skin, sputum, nasopharangeal aspirates, and swabs, including endocervical, vaginal, occular, throat and buccal swabs, hair, cerebrospinal fluid or other body fluids, including tissues, cell cultures and cell suspensions.

The composition and method of the invention provide advantages over prior known compositions and methods including more rapid and more economical processing and the use of chemical rather than enzymatic treatment. The composition and method of the invention also permit the use of a higher sample volume. Prior methods required dilution of a sample such as plasma by as much as 50% before enzyme digestion. In contrast, the present invention permits extraction of nucleic acid from 100% plasma using paramagnetic particles. The present invention also permits the drying down of reagents, which can then be easily stored in tubes and remain stable for long periods of time.

The composition of present invention denatures proteins and lyses infectious agents such as viruses and bacteria during nucleic acid extraction. It is believed that the combination of alkaline agent and detergent inactivates RNases/DNases, which would otherwise hinder the extraction of nucleic acid. The present invention is thus directed to a composition that comprises at least one alkaline agent and at least one detergent. The detergents that are useful for the present invention include anionic, nonionic and zwitterionic detergents. Suitable anionic detergents include, but are not limited to, sodium dodecyl sulfate and lithium dodecyl sulfate. Suitable nonionic detergents include, but are not limited to, polyethylene glycol sorbitan monolaurate (i.e., Tween® 20), polyethylene glycol sorbitan monooleate (i.e., Tween® 80), NP-40, polyethylene glycol tert-octylphenyl ether (i.e., Triton X detergents such as Triton X-20 and Triton X-100) and cetyl trimethyl ammonium bromide (CTAB). Suitable zwitterionic detergents include, but are not limited to, 3[(3-cholamidopropyl)dimethylammonio]-1-propanesulfonate (CHAPS) and other zwitterionic surfactants. The alkaline agents useful in the present invention include, but are not limited to, bases and alkaline buffers such as KOH, NaOH, NH₄OH and Ca(OH)₂, phosphate buffers, saline buffers, borate buffers, Tris buffer and the like.

The composition according to the present invention comprises an alkaline agent in an amount of about 10 mM to about 400 mM and a detergent in an amount of about 0.05% to about 10% by volume. Preferably, the composition contains about 100 mM to about 200 mM of an alkaline agent and about 0.1% to about 3.0% by volume of a detergent.

According to one embodiment, the composition is in a liquid solution and is placed in a container. According to another embodiment, one or more of the components of the composition, alone or in combination, may be dried by methods, such as vacuum drying or freeze drying, that are known in the art. For example, by freezing the solution and then slowly warming after freezing, while simultaneously applying a vacuum, a freeze-dried powder remains in the container, e.g., an extraction tube or a blood collection tube. An additive such as an excipient, for example, polyvinyl-pyrrolidone (“PVP”) or trehalose, may also be added as a stabilizing agent to the solution prior to drying so that the resulting stabilizing agent is dried in the container. In another aspect, the composition, or a subset of the composition, is formed into a liquid or suspension and is dispersed or sprayed onto one or more surfaces of the interior of the container.

The composition preferably includes a suspension of paramagnetic particles. Iron particles are useful as the paramagnetic particles in the present invention, and the iron may be an iron oxide of forms such as ferric hydroxide or ferrosoferric oxide. Other iron particles such as iron sulfide and iron chloride may also be suitable for binding and extracting nucleic acids using the conditions described herein.

The shape of the paramagnetic particles is not critical to the present invention. The paramagnetic particles may be of various shapes including, for example, spheres, cubes, ovals, capsule-shaped, tablet-shaped, nondescript random shapes, etc., and may be of uniform shape or non-uniform shape. Whatever the shape of a paramagnetic particle, its diameter at its widest point is generally in the range of from about 0.05 to about 20.0 microns.

The concentration of the particles may vary depending on the biological sample. For most biological samples, the concentration of the paramagnetic particles is about 1 mg/mL to about 500 mg/mL. However, diluted sample or concentrated samples may need less or more paramagnetic particles.

The composition of the present invention may further comprise an acidic solution such as acids and acidic buffer solutions. The acidic solution in combination with the paramagnetic particles may then be used to extract the nucleic acid from proteinaceous samples without clotting the sample. Any acid may be used. Exemplary acids include, but are not limited to, phosphoric acid, nitric acid, sulfuric acid, acetic acid and citric acid.

The acidic environment in which the paramagnetic particles effectively and reversibly bind nucleic acid molecules can be provided through a variety of means. For example, the paramagnetic particles can be added to an acidic solution or an acidic solution may be added to the particles. Alternatively, a solution or environment in which the paramagnetic particles are located can be acidified by addition of an acidifying agent. The acid is sufficient to bring the pH of the alkaline agent/detergent composition to an acidic pH, i.e., between about 1 to about 7.

As stated above, in an acidic environment, electropositive paramagnetic particles will bind electronegative nucleic acid molecules. Other materials in the environment, such as inhibitors of nucleic acid hybridization and amplification can, therefore, be separated from the bound nucleic acid molecules. Such separation can be accomplished by means known to those skilled in the art, such as centrifugation, filtering or application of magnetic force.

The bound nucleic acid molecules can then be eluted into an appropriate buffer for further manipulation, such as hybridization or amplification reactions. Such elution can be accomplished by heating the environment containing the particles with bound nucleic acids and/or raising the pH of such environment. Agents which can be used to aid the elution of nucleic acid from the paramagnetic particles include water, buffers, alkaline agents such as KOH, NaOH, NH₄OH and Ca(OH)₂, phosphate buffers, saline buffers, borate buffers, Tris buffer or any compound that increases the pH of the environment to an extent sufficient that electronegative nucleic acid is displaced from the particles.

The reversible binding and elution of nucleic acids on paramagnetic particles is primarily achieved by altering the pH of the media, in which the binding and elution procedures take place following the alkaline agent/detergent pre-treatment. Nucleic acids bind onto the surface of these particles in acidic pH and elute at neutral or alkaline pH. However, there are other factors that also affect the binding and elution. For example, reduced temperature may increase binding of nucleic acids on the solid surface and increased temperature may enhance the elution process. The concentration of detergent used for the pre-treatment may also play a role in nucleic acid binding. The combination of detergent and alkaline agent treatment presumably (1) denatures proteins such as DNases and RNases and (2) lyses cells and/or microorganisms such as virions and/or bacterial cells. The concentrations of detergent and alkaline agent should be high enough to disrupt the walls or membranes of cells and virions, denature proteinaceous material and solubilize the targeted nucleic acids.

The following example illustrates specific embodiments of the invention. As would be apparent to skilled artisans, various changes and modifications are possible and are contemplated within the scope of the invention described.

### EXAMPLE

The following example demonstrates the use of chemical treatment and compares such treatment to enzymatic digestion during extraction of HIV RNA from plasma. The efficiency of RNA extraction was evaluated using an HIV SDA assay.

Treatment

1. Dispense 22 mL of human plasma and 13.2 mL of 30 mM KPO₄ (pH 7.6) into a 50-mL tube.

2. Add 80 μg/mL of yeast carrier RNA into the tube.

3. Spike the human plasma with HIV particles at the level of 1000 particles/mL.

4. Mix well and dispense 16 mL of plasma mixture into two tubes (tubes A and B).

5a. In tube A, add 1.1 mL of Proteinase K (600 units/mL), mixing well by inverting tube 6 times.

5b. Incubate tube A in 70° C. waterbath for 30 minutes.

5c. Transfer 850 μL of plasma mixture into 2-mL extraction tubes containing 40 mg of iron oxide.

5d. Mix by inverting tubes in 5-minute interval.

6a. Dispense 800 μL of plasma mixture from tube B into 16 extraction tubes containing 40 mg of iron oxide, 100 μmoles KOH, and 10 μL of Triton (these chemicals were dried down in tubes).

6b. Mix well by inverting tubes 6 times.

6c. Incubate 8 tubes in a 70° C. waterbath for 30 minutes.

6d. Incubate another 8-tube set at room temperature for 30 minutes.

6e. Mix by inverting tubes in 5-minute interval.

7. Allow all tubes to cool down at room temperature for another 30 minutes before extraction.

Binding and Elution

1. Dispense 270 μL of Binding acid (either 6 M glycine.HCl or 6 M H₃PO₄) into tubes and mix 25 times.

2. Magnetically lock iron oxide particles to the sides of tubes.

3. Aspirate the unbound sample.

4. Wash the iron oxide particles with 1020 μL of 1 mM of glycine.HCl or 1 mM H₃PO₄.

5. Magnetically lock the iron oxide particles and aspirate the unbound solution.

6. Wash iron oxide particles with 1020 μL of 1 mM of glycine.HCl or 1 mM H₃PO₄.

7. Magnetically lock the iron oxide particles and aspirate the unbound solution.

8. Dispense 120 μL elution buffer (85 mM KOH/75 mM Bicine) into the tube and mix 15 times.

9. Magnetically lock the iron oxide particles and aspirate the unbound solution.

10. Dispense 60 μL of neutralization buffer (460 mM Bicine) into the tube and mix the sample 15 times.

11. Magnetically lock the iron oxide particles and aspirate the unbound solution.

12. The eluted samples are ready for SDA assay (50 μL of sample per assay).

| Sample | Plasma | Plasma | Plasma | Plasma | Assay | Assay |
|---|---|---|---|---|---|---|
| | | | | | buffer | buffer |
| Target | HIV | HIV | HIV | HIV | RNA | RNA |
| | particles | particles | particles | particles | transcripts | transcripts |
| (copies/rxn) | 200 | 200 | 200 | 200 | 50 | 0 |
| Treatment | ProK | ProK | KOH/Triton | KOH/Triton | NA | NA |
| Temperature | 70° C. | 70° C. | 70° C. | RT | NA | NA |
| Binding Acid | Glycine.HCl | H₃PO₄ | H₃PO₄ | H₃PO₄ | Pos. Ctl. | Neg. Ctl. |
| | 16288 | 15082 | 21231 | 25877 | 54900 | 31 |
| | 15172 | 36351 | 22749 | 33803 | 34578 | 29 |
| | 14140 | 31430 | 21044 | 33007 | 47591 | 0 |
| | 18146 | 35206 | 24992 | 33360 | 60299 | 10 |
| | 18466 | 38333 | 37972 | 32719 | 62607 | 27 |
| | 7656 | 38215 | 35586 | 37108 | 61437 | 26 |
| | 19799 | 34641 | 33518 | 31997 | 49383 | 30 |
| | 8220 | 33189 | 27612 | 36132 | 60305 | 29 |
| Average | 14736 | 32806 | 28088 | 33000 | 53888 | 23 |
| SD | 4282 | 7051 | 6308 | 3148 | 9008 | 11 |
| CV % | 29 | 21 | 22 | 10 | 17 | 47 |

The results demonstrate that RNA can be extracted from HIV particles in plasma treated using an alkaline agent and a detergent according to the present invention. The extracted RNA can be used in nucleic acid amplification assays such as SDA. The combination of the chemical treatment of plasma and use of phosphoric acid as a binding acid had equivalent or better results than use of the enzyme digestion method.

The method of the invention is advantageous in that it does not rely on the use of enzymes. It is therefore less expensive and would generally be expected to be a more robust process. It also has the added advantage of being effective at room temperature and not requiring extended periods of time for incubation. The process would also be expected to be applicable to DNA and RNA extraction from a number of biological samples.

Note that this experiment utilized diluted plasma (62.5%). Attempts to utilize 100% plasma treated using the protease method resulted in sample coagulation during enzyme treatment, thereby rendering nucleic acid extraction extremely difficult and inefficient. However, use of the chemical “no protease” method of the invention improves sample solubility throughout the nucleic acid extraction process, thereby allowing extraction from a greater percentage of sample and, hence, greater sensitivity of detection.

As discussed herein, the volume of the sample can be varied. For example, the sample can be diluted with buffers such as 30 mM potassium phosphate before or after treatment. Incubation time and temperature can also be varied. The concentration and type of alkaline agent and detergent can be varied. The protocol can be used with manual or automated nucleic acid extraction methods. The type and concentration of acid can be varied. The extracted nucleic acids can be used for a variety of down stream applications.

While the invention has been described with some specificity, modifications apparent to those with ordinary skill in the art may be made without departing from the scope of the invention. Various features of the invention are set forth in the following claims.

1. A method for purifying nucleic acid from a biological sample, comprising:
(a) treating said biological sample with at least one alkaline agent and at least one detergent;
(b) providing a suspension of at least one paramagnetic particle;
(c) providing an acidic solution; and
(d) combining said suspension and said acidic solution with said treated biological sample such that at least one nucleic acid molecule in said biological sample is reversibly bound to said at least one paramagnetic particle.

2. The method of claim 1, wherein said at least one paramagnetic particle comprises iron.

3. The method of claim 1, wherein said at least one paramagnetic particle is selected from the group consisting of an iron oxide, iron sulfide and iron chloride.

4. The method of claim 3, wherein the iron oxide is selected from the group consisting of ferric hydroxide and ferrosoferric oxide.

5. The method of claim 1 further comprising:
(e) eluting said at least one nucleic acid molecule from said at least one paramagnetic particle.

6. The method of claim 5, wherein said eluting comprises contacting said reversibly bound nucleic acid with a reagent selected from the group consisting of Tris, Bicine, CAPS, HEPES, water, potassium phosphate, Tricine, and assay buffers.

7. The method of claim 5 further comprising:
(f) detecting said at least one nucleic acid molecule.

8. The method of claim 5 further comprising:
(f) amplifying said at least one nucleic acid molecule after eluting.

9. The method of claim 1, wherein the alkaline agent is selected from the group consisting of KOH, NaOH, NH₄OH and Ca(OH)₂.

10. The method of claim 1, wherein the detergent is selected from the group consisting of anionic, nonionic and zwitterionic detergents.

11. The method of claim 10, wherein the anionic detergent is selected from the group consisting of sodium dodecyl sulfate and lithium dodecyl sulfate.

12. The method of claim 10, wherein the nonionic detergent is selected from the group consisting of polyethylene glycol sorbitan monolaurate, polyethylene glycol sorbitan monooleate, NP-40, polyethylene glycol tert-octylphenyl ether and cetyl trimethyl ammonium bromide.

13. The method of claim 10, wherein the zwitterionic detergent is selected from the group consisting of 3[(3-cholamidopropyl)dimethylammonio]-1-propanesulfonate and zwitterionic surfactants.

14. The method of claim 1, wherein said alkaline agent and said detergent are added to bring said biological sample to a pH of about 7 to about 12.

15. The method of claim 1, further comprising extracting said nucleic acid with an acid selected from the group consisting of phosphoric acid, nitric acid, hydrochloric acid, sulfuric acid, acetic acid and citric acid.

16. The method of claim 1, wherein potassium phosphate is employed.

17. The method of claim 1, wherein said nucleic acid is selected from the group consisting of DNA and RNA.

18. The method of claim 1, wherein said biological sample is selected from the group consisting of whole blood, plasma, serum, urine, semen, feces, finger nails, skin, sputum, nasopharangeal aspirates, and swabs, including endocervical, vaginal, occular, throat and buccal swabs, hair, cerebrospinal fluid, tissue, cell culture and cell suspension.

19. A composition for purifying nucleic acid from a biological sample, the composition comprising
at least one alkaline agent;
at least one detergent; and
at least one paramagnetic particle;
wherein the at least one alkaline agent and the at least one detergent are present in concentrations that can denature proteins and lyse infectious agents.

20. The composition of claim 19, wherein the composition comprises a suspension of the at least one paramagnetic particle.

21. The composition of claim 20, further comprising an acidic solution.

22. The composition of claim 19, wherein said alkaline agent and said detergent are in a liquid solution.

23. The composition of claim 19, wherein said alkaline agent and said detergent are in dry form.

24. The composition of 19, wherein the alkaline agent is KOH and the detergent is polyethylene glycol tert-octylphenyl ether.

25. A kit for purifying nucleic acid from a biological sample, the kit comprising:
at least one alkaline agent;
at least one detergent; and
at least one paramagnetic particle;
wherein the at least one alkaline agent and the at least one detergent are present in concentrations that can denature proteins and lyse infectious agents.

26. The kit of claim 25, wherein the kit comprises a suspension of at least one paramagnetic particle.

27. The kit of claim 26, further comprising an acidic solution.

28. The kit of claim 24, wherein the alkaline agent is KOH and the detergent is polyethylene glycol tert-octylphenyl ether.

29. The method of claim 5, wherein said eluting is conducted at a pH of about 7 to about 12.

30. The method of claim 1, wherein said alkaline agent and said detergent bring said biological sample to a pH of about 7 to about 12 in step (a).

31. The method of claim 1, wherein said acidic solution brings said biological sample/alkaline agent/detergent to a pH of about 1 to about 7 in step (c).

32. The composition of claim 19, wherein said alkaline agent is present in an amount from about 10 mM to about 400 mM.

33. The composition of claim 32, wherein said detergent is present in an amount from about 0.05% to about 10.0% by volume.

34. The method of claim 8, wherein said method is performed in a single vessel.

35. The method of claim 34, wherein said providing a suspension of at least one paramagnetic particle comprises adding said biological sample to said single vessel.

36. A composition for purifying nucleic acid from a biological sample, the composition comprising:
at least one alkaline agent;
at least one detergent; and
at least one paramagnetic particle;
wherein the at least one alkaline agent is present in the composition in an amount from about 10 mM to about 400 mM, and the at least one detergent is present in an amount from about 0.05% to about 10.0% by volume.

37. A kit comprising the composition of claim 36.

38. The composition of claim 36, wherein the alkaline agent is KOH and the detergent is polyethylene glycol tert-octylphenyl ether.

39. The composition of claim 36, wherein the alkaline agent is KOH and the detergent is polyethylene glycol tert-octylphenyl ether.
